(19) United States
(12) Patent Application Publication
(10) Pub. No.: US 2009/0182240 A1
(43) Pub. Date: Jul. 16, 2009

(54) APPARATUS AND SENSOR FOR MEASURING BIOLOGICAL SIGNAL AND APPARATUS AND METHOD FOR MEASURING PULSE WAVE VELOCITY

(75) Inventors: Woo-young Jang, Seongnam-si (KR); Sang-kon Bae, Seongnam-si (KR); Kun-soo Shin, Seongnam-si (KR)

Correspondence Address: SUGHRUE MION, PLLC, 2100 PENNSYLVANIA AVENUE, N.W., SUITE 800, WASHINGTON, DC, 20037, US

(73) Assignee: Samsung Electronics Co., Ltd., Suwon-si (KR)

(21) Appl. No.: 12/060,940

(22) Filed: Apr. 2, 2008

(30) Foreign Application Priority Data
Jan. 16, 2008 (KR) 10-2008-0004907

(51) Int. Cl. A61B 5/026

(52) U.S. Cl. 600/504

(57) ABSTRACT

Provided is an apparatus for measuring a biological signal. The apparatus includes a first surface having a first sensor which is attached to a predetermined part of a user's body and measures a first biological signal generated by the predetermined part; and a second surface having a second sensor which is attached to the user's finger and measures a second biological signal generated by the finger. Therefore, a user can easily check his or her health condition without being limited by time or place.

### CROSS-REFERENCE TO RELATED PATENT APPLICATION

This application claims the priority of Korean Patent Application No. 10-2008-0004907, filed on Jan. 16, 2008, in the Korean Intellectual Property Office, the disclosure of which is incorporated herein in its entirety by reference.

### BACKGROUND

1. Field

One or more embodiments of the present invention relate to an apparatus and sensor for measuring a biological signal, and more particularly, to an apparatus and sensor for measuring a biological signal, and an apparatus, method, and medium for measuring a pulse wave velocity (PWV).

2. Description of the Related Art

In order to sustain life, blood ejected from the heart with each heartbeat must freely flow from the heart to each part of the human body through the arteries and flow back to the heart through the veins. In so doing, oxygen and nutrients can be supplied to every tissue of the human body, and body wastes produced after metabolism can be removed.

However, if blood is not properly delivered to a certain part of the body since the arteries are unhealthy or if the blood is thick due to blood clots or emboli being generated within the blood, capillary vessels of a certain tissue of the body may be clogged, thereby causing necrosis of the tissue. Therefore, life can be threatened by a disease such as a stroke, which occurs when blood clots generated in the carotid artery block the bloodstream in the brain, diabetes, a diabetic foot, impaired kidney, or myocardial infarction which occurs when the coronary arteries are clogged.

It is reported that circulatory diseases is the second most common cause of death in Korea after cancer and that arteriosclerosis accounts for approximately 90% of the circulatory diseases. Arteriosclerosis refers to the thickening, hardening and loss of elasticity of arterial walls and is a major cause of high blood pressure, obesity and diabetes. In addition, arteriosclerosis causes bloodstream troubles, generation of blood clots, strokes, and myocardial infarction. In this regard, it is extremely important to diagnose and prevent cardiovascular diseases and arteriosclerosis at an early stage.

Methods of diagnosing cardiovascular diseases and arteriosclerosis are classified into invasive methods and non-invasive methods. The invasive methods may include angiography, in which blood vessels are X-rayed by a contrast medium injected into the blood vessels, a method using a catheter, and ultrasonography of the arteries.

The non-invasive methods may include imaging diagnosis using magnetic resonance imaging (MRI), computer tomography (CT) or ultrasonic waves, a method of measuring a pulse wave velocity (PWV), and a method of measuring an augmentation index (AI) which indicates variations in level of pulse pressure according to reflected waves. Recently, non-invasive methods have widely been used to diagnose the condition of blood vessels.

A person's pulse is the throbbing of their arteries as an effect of the heart beat. A pulse wave is a waveform of pulsation of the peripheral venous and arterial system which occurs at the same time as the systole or diastole. A PWV refers to the speed at which a pulse wave passes through an arterial vessel. For example, the PWV can be calculated by dividing the distance between two locations of a blood vessel, where a pulse wave is detected, by the difference between points of time when the pulse wave is detected at the two locations. If an arterial vessel hardens, the PWV is increased. Therefore, the PWV is used as a quantitative index of arteriosclerosis.

### SUMMARY

One or more embodiments of the present invention provide an apparatus for measuring a biological signal, the apparatus enabling a user to easily check his or her health condition without being limited by time or place.

One or more embodiments of the present invention also provide a sensor for measuring a biological signal, the sensor enabling a user to easily check his or her health condition without being limited by time or place.

One or more embodiments of the present invention also provide an apparatus and method for measuring a pulse wave velocity (PWV), the apparatus and method enabling a user to easily check his or her health condition without being limited by time or place, and a computer-readable medium having computer readable code to implement the method.

Additional aspects and/or advantages will be set forth in part in the description which follows and, in part, will be apparent from the description, or may be learned by practice of the invention.

According to an aspect of the present invention, there is provided an apparatus for measuring a biological signal, the apparatus including a first surface having a first sensor which is attached to a predetermined part of a user's body and measures a first biological signal generated by the predetermined part; and a second surface having a second sensor which is attached to the user's finger and measures a second biological signal generated by the finger.

According to another aspect of the present invention, there is provided a biological signal measuring sensor, the biological signal measuring sensor including a first sensor to be attached to the skin near the heart of a user and measure the systole and diastole caused by a heartbeat; and a second sensor to be attached to the user's finger and measure changes in the blood volume in blood vessels near the finger which are caused by a heartbeat, wherein the first and second sensors are integrated in the biological signal measuring sensor.

According to another aspect of the present invention, there is provided an apparatus for measuring a PWV, the apparatus including a biological signal measuring sensor in which a first sensor and a second sensor are integrated; an input unit to receive a user's body information; and a calculating unit to calculate a PWV according to the systole and diastole of the user by using measurement results of the biological signal measuring sensor and the body information input to the input unit, wherein the first sensor is attached to the skin near the heart of the user and measures the systole and diastole caused by a heartbeat, and the second sensor is attached to the user's finger and measures changes in the blood volume in blood vessels near the finger which are caused by a heartbeat.

According to another aspect of the present invention, there is provided a method of measuring a PWV, the method including measuring a point of time when blood is ejected from the heart of a user and a point of time when the blood arrives at a specified tissue by using a biological signal measuring sensor in which a plurality of sensors are integrated; inputting body information of the user; and calculating a PWV according to the systole and diastole of the user by using measurement results and the input body information.

According to another aspect of the present invention, there is provided a computer-readable medium having a computer readable code to implement a method of measuring a PWV, the method including: measuring a point of time when blood is ejected from the heart of a user and a point of time when the blood arrives at a specified tissue by using a biological signal measuring sensor in which a plurality of sensors are integrated; inputting body information of the user; and calculating a PWV according to the systole and diastole of the user by using measurement results and the input body information.

### BRIEF DESCRIPTION OF THE DRAWINGS

These and/or other aspects and advantages will become apparent and more readily appreciated from the following description of the embodiments, taken in conjunction with the accompanying drawings of which:

FIG. 1 is a block diagram of an apparatus for measuring a pulse wave velocity (PWV) according to an embodiment of the present invention;

FIGS. 2A and 2B are diagrams for explaining the operation of a calculating unit shown in FIG. 1;

FIG. 3 shows an example of the apparatus of FIG. 1 in which a plurality of sensors are integrated;

FIG. 4 shows an example of the apparatus of FIG. 3 in which a plurality of sensors are integrated;

FIG. 5 shows an example of a photoplethysmography (PPG) sensor included in the apparatus of FIG. 4;

FIG. 6 shows an example of a phonocardiogram (PCG) sensor included in the apparatus of FIG. 4;

FIG. 7 shows waveforms of a PPG and a PCG measured by the apparatus of FIG. 4; and

FIG. 8 is a flowchart illustrating a method of measuring a PWV according to an embodiment of the present invention.

### DETAILED DESCRIPTION OF THE EMBODIMENTS

Reference will now be made in detail to embodiments, examples of which are illustrated in the accompanying drawings, wherein like reference numerals refer to the like elements throughout. In this regard, embodiments of the present invention may be embodied in many different forms and should not be construed as being limited to embodiments set forth herein. Accordingly, embodiments are merely described below, by referring to the figures, to explain aspects of the present invention.

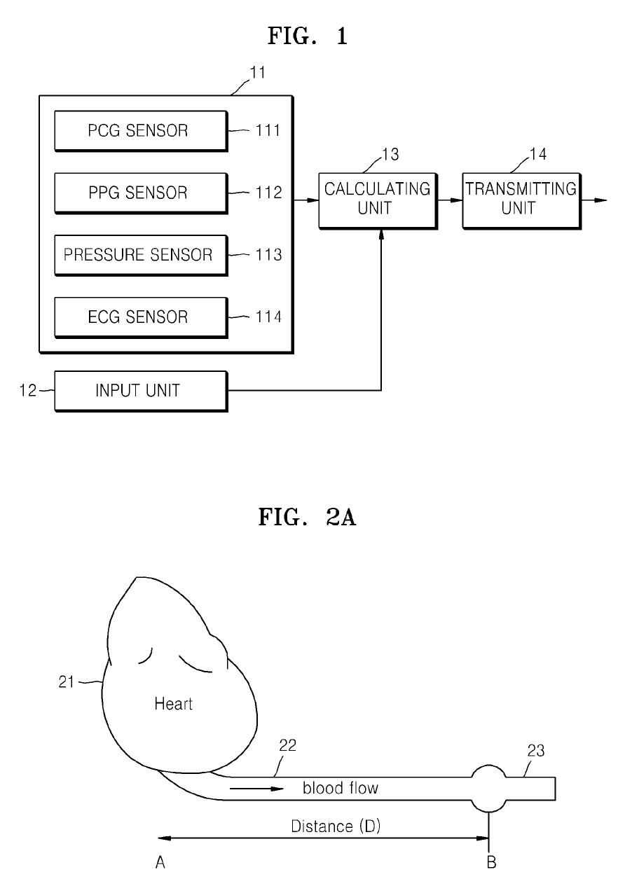

FIG. 1 is a block diagram of an apparatus for measuring a pulse wave velocity (PWV) according to an embodiment of the present invention.

Referring to FIG. 1, the apparatus may include a biological signal measuring sensor 11, an input unit 12, a calculating unit 13, and a transmitting unit 14.

The biological signal measuring sensor 11 may be attached to a human body and sense various biological signals generated by the human body. Specifically, the biological signal measuring sensor 11 may include a phonocardiogram (PCG) sensor 111, a photoplethysmography (PPG) sensor 112, and a pressure sensor 113. The biological signal measuring sensor 11 may further include an electrocardiogram (ECG) sensor 114.

The PCG sensor 111 may be attached to the skin near the heart and sense a PCG. The PCG is a record of heart sounds generated when four valves of the heart are opened or closed and heart murmurs generated when the stroma of the heart is changed. Heart sounds are classified into audible first and second heart sounds and inaudible third and fourth heart sounds. Specifically, the first heart sound is generated when atrioventricular valves, which prevent the backflow of blood between atria and ventricles, are closed. The second heart sound is generated when an aortic valve, which prevents the backflow of blood between the left ventricle and the aorta, is closed. The third heart sound is generated when the atrioventricular valves are opened, and the fourth heart sound is generated when the ventricles contract. The first through fourth heart sounds are sequentially and repeatedly generated in each cardiac cycle which is the time taken from the beginning of the contraction of the atria to the beginning of the next contraction. A systolic phase of the cardiac cycle is between the first and second heart sounds, and a diastolic phase thereof is between the second heart sound and a next first heart sound.

The PPG sensor 112 may be attached to a fingertip or the tip of a toe and measure a PPG in a specified tissue. Specifically, the PPG sensor 112 may input infrared light to a specified tissue and measure the intensity of the infrared light absorbed by the specified tissue to detect a change in the blood volume in the specified tissue. The PPG sensor 112 takes advantage of the property of red blood cells in blood to absorb infrared light. In particular, the PPG sensor 112 measures the blood volume flowing into capillary vessels via arteries and arterioles and checks whether blood normally flows to peripheral parts of the human body, such as fingertips and the tip of the toes.

Since the PPG fluctuates according to the phase of the cardiac cycle, the phase of the cardiac cycle can be determined based on the measurement of the PPG. Specifically, in the systolic phase of the cardiac cycle, blood ejected from the left ventricle of the heart is transported to each tissue of the peripheral parts (such as fingertips and the tip of the toes) of the human body. Accordingly, the volume of blood in the arteries is increased, which, in turn, increases the intensity of light absorbed by each tissue. In the diastolic phase, some of the blood in each tissue of the peripheral parts of the human body flows into the heart. Thus, the intensity of light absorbed by each tissue is reduced. Since the blood volume slightly changes according to the phase of the cardiac cycle as described above, the intensity of light absorbed by each tissue, that is, changes in absorbance, can be measured by the PPG sensor 112.

The pressure sensor 113 may measure the force applied by the hand to grip the apparatus for measuring a PWV, that is, grip strength. The apparatus according to the present embodiment may be pen-shaped or stethoscope-shaped. When a user grips the apparatus of the pen shape by the hand, noise may be generated by the pressure applied to the apparatus.

Therefore, the pressure sensor 113 measures the force applied by the hand to grip the apparatus in real time in order to keep the force higher than a predetermined force. For example, the apparatus may operate only when a force greater than the predetermined force is applied to grip the apparatus.

The ECG sensor 114 may measure an ECG. Specifically, the ECG measures variations in the action potential of heart muscle cells, which are caused by heartbeats, over time and graphically represents the measured variations. The ECG sensor 114 may be attached to the skin near the heart or to each arm or hand in order to measure heartbeats and sense the ECG.

Conventional PCG, PPG and ECG sensors are discrete and independent devices and have been used to measure the PCG, PPG and ECG of a person, respectively. In addition, a separate processing apparatus has been used to measure the PWV of the person based on the measured PCG, PPG and ECG. That is, since a number of devices are required to measure PWV, people had to visit medical centers to measure their PWVs.

However, a PCG sensor and a PPG sensor may be integrated into a biological signal measuring sensor included in an apparatus for measuring a PWV according to an embodiment of the present invention. Alternatively, the biological signal measuring sensor may further include a pressure sensor. Thus, the PCG sensor, the PPG sensor and the pressure sensor may be integrated into the biological signal measuring sensor. Alternatively, a PPG sensor, a pressure sensor, and an ECG sensor may be integrated into a biological signal measuring sensor included in an apparatus for measuring a PWV according to another embodiment of the present invention. Alternatively, a PCG sensor, a PPG sensor, a pressure sensor, and an ECG sensor may be integrated into a biological signal measuring sensor included in an apparatus for measuring a PWV according to another embodiment of the present invention.

Since a plurality of necessary sensors are integrated into a biological signal measuring sensor according to embodiments of the present invention, a user can carry the biological signal measuring sensor and check the condition of his or her blood vessels without being limited by time or place. Therefore, the user can recognize the influence of certain foods or exercises on the condition of his or her blood vessels.

The input unit 12 may receive body information of a user. Specifically, the input unit 12 may be implemented outside the apparatus and used by a user to input his or her body information such as age, gender, height and weight.

The calculating unit 13 may calculate the PWV based on outputs of the biological signal measuring sensor 11 and the input unit 12, which will now be described in detail.

First, the calculating unit 13 may calculate a pulse transit time (PTT) by using the PCG, the PPG and ECG sensed by the biological signal measuring sensor 11. The PTT is between a first point of time when blood is ejected from the heart and a second point of time when the blood ejected from the heart arrives at a specified tissue. The first point of time when blood is ejected from the heart is calculated based on the PCG or the ECG. The PCG peaks when the first and second heart sounds are generated, and the same pattern applies to the ECG. A point of time when the PCG or ECG reaches its peak, which indicates the generation of the first heart sound, corresponds to the first point of time when blood is ejected from the heart. In addition, the second point of time when the blood ejected from the heart arrives at a specified tissue is calculated using the PPG. The PPG begins to peak when the blood arrives at the specified tissue.

Next, the calculating unit 13 may calculate the distance traveled by blood, which is ejected from the heart, by using the body information of the user input to the input unit 12. The distance traveled by blood is the distance between the heart, from which the blood is ejected, and each tissue at which the PPG is measured. The distance traveled by blood can be calculated based on the height of the user, which is input to the input unit 12. For example, the calculating unit 13 may calculate the distance traveled by blood based on the height of the user by referring to a database of the height of the user and the distance between the heart and each tissue of the user.

Then, the calculating unit 13 may calculate the PWV by dividing the calculated distance by the PTT. The calculated PWV is used to diagnose the condition of the user's blood vessels and determine how much arteriosclerosis the user has developed.

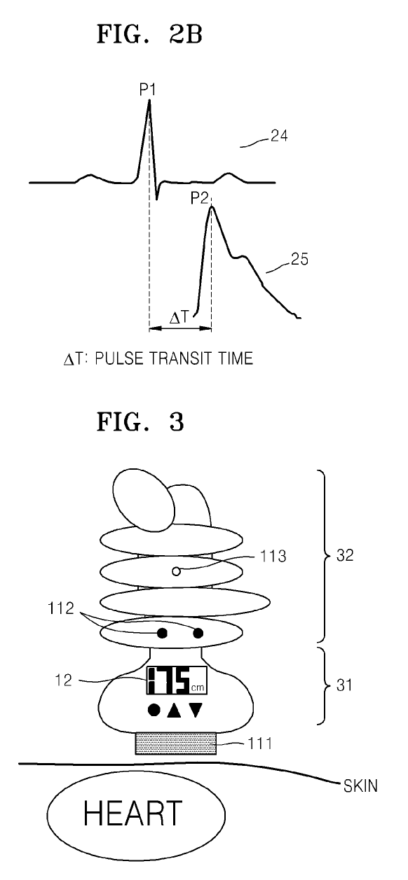

FIGS. 2A and 2B are diagrams for explaining the operation of the calculating unit 13 shown in FIG. 1. A process in which the calculating unit 13 calculates the PWV will now be described with reference to FIGS. 1 and 2A and 2B.

Referring to FIG. 2A, reference numeral 21 indicates the heart, reference numeral 22 indicates an artery, and reference numeral 23 indicates a peripheral blood vessel. A distance D traveled by blood is from a position A of the heart 21 to a position B of a specified tissue.

FIG. 2B is a waveform of an ECG 24 and a PPG 25. Referring to FIG. 2B, a difference ΔT between a point of time when the ECG 24 reaches its peak P1 and a point of time when the PPG 25 reaches its peak P2 is the time taken by blood ejected from the heart to arrive at the specified tissue, that is, the PTT. If a waveform of a PCG, instead of the ECG 24, is used, the PTT can be calculated using a point of time when the generation of the first heart sound is detected in the PCG.

The calculating unit 13 divides the distance D traveled by blood, which was calculated as shown in FIG. 2A, by the PTT calculated as shown in FIG. 2B to obtain a PWV indicating the velocity at which a pulse wave passes through arterial vessels.

Referring back to FIG. 1, the transmitting unit 14 may transmit, in a wired or wirelessly manner, a PWV calculated by the calculating unit 13 to an external device. For example, the transmitting unit 14 may transmit a PWV calculated by the calculating unit 13 to a medical center. Thus, the medical center can remotely diagnose the condition of a patient's blood vessels.

Although not shown in FIG. 1, the apparatus for measuring a PWV according to the present embodiment may further include a display unit. In this case, the display unit may display a PWV calculated by the calculating unit 13 so that a user can check the condition of his or her blood vessels.

FIG. 3 shows an example of the apparatus of FIG. 1 in which a plurality of sensors are integrated.

Referring to FIG. 3, the apparatus may include a first member 31 and a second member 32. The first member 31 includes a first sensor which is attached to the skin near the heart and measures the systole and diastole caused by a heartbeat. The second member 32 includes a second sensor which is attached to a user's finger and measures changes in the blood volume in blood vessels near the finger which are caused by a heartbeat. The first member 31 may be plate-shaped, and the second member 32 may be pole-shaped.

Specifically, the apparatus may be implemented as a pole of a predetermined shape, wherein the pole has a bottom surface which contacts the skin near the heart and a side surface which can be held by the hand. More specifically, the apparatus may be implemented as a cylindrical or polygonal pole. In this case, finger grooves may be formed on a cylindrical surface of the apparatus so that the apparatus can be easily gripped by the hand. For example, the apparatus may be pen-shaped or stethoscope-shaped.

However, the apparatus shown in FIG. 3 is merely an embodiment of the present invention, and the present invention is not limited thereto. It will be understood by those of ordinary skill in the art that there are many variations of the configuration of an apparatus for measuring a PWV according to another embodiment of the present invention, the apparatus including a first sensor and a second sensor which are integrated with each other. As described above, the first sensor is attached to the skin near the heart and measures the systole and diastole caused by a heartbeat. In addition, the second sensor is attached to a peripheral part, such as a user's finger, and measures changes in the blood volume in blood vessels near the peripheral part which are caused by a heartbeat.

More specifically, an apparatus for measuring a PWV according to another embodiment of the present invention may include a first surface having a first sensor, which is attached to the skin near the heart and measures the systole and diastole caused by a heartbeat, and a second surface having a second sensor which is attached to a user's finger and measures changes in the blood volume in blood vessels near the finger which are caused by a heartbeat. For example, the apparatus according to another embodiment of the present invention may be a hexahedron having a first surface, which contacts the skin near the heart, and a second surface which contacts a finger. Alternatively, an apparatus for measuring a PWV according to another embodiment of the present invention may be of a semi-cylindrical shape having a flat first surface, which contacts the skin near the heart, and a curved second surface which contacts a finger.

The PCG sensor 111 may be mounted on a bottom surface of the first member 310 of the apparatus shown in FIG. 1. The PCG sensor 111 may contact the skin near the heart and measure the PCG. Although not shown in FIG. 3, an ECG sensor may be mounted next to the PCG sensor 111. In this case, the ECG sensor may be mounted on the bottom surface of the first member 310 in a two-point or three-point fashion.

Specifically, the apparatus according to the present embodiment may have a flat bottom surface so that the PCG sensor 111 or the ECG sensor (not shown) can be attached to the skin near the heart as closely as possible in order to measure the PCG or the ECG with increased accuracy. Since the PCG sensor 111 or the ECG sensor (not shown) is mounted on the bottom surface of the apparatus, a point of time when blood is ejected from the heart as the heart contracts can be measured.

In addition, the input unit 12 may be mounted on the side surface of the first member 31 of the apparatus, that is, a cylindrical surface of the apparatus. Thus, a user can directly input his or her body information, such as height and age, to the input unit 12. However, in another embodiment of the present invention, the input unit 12 may be mounted on a side surface of the second member 32 of the apparatus.

The PPG sensor 112 may be mounted on the side surface of the second member 32 in a two-point fashion. Since the PPG sensor 112 is mounted in a two-point fashion, one point may irradiate infrared light to the skin of a user, and the other point may measure the amount of infrared light reflected off the skin of the user. In addition, the pressure sensor 113 may be mounted on the side surface of the second member 32 of the apparatus. Thus, changes in the PPG caused by the grip strength of a user can be compensated for.

Specifically, finger grooves may be formed on the side surface of the second member 32 of the apparatus according to the present embodiment, so that a user can easily grip the apparatus. In addition, the PPC sensor 112 and the pressure sensor 113 may be mounted in the finger grooves on the side surface of the second member 32, so that the PPC sensor 112 and the pressure sensor 113 can measure the PPC and grip strength of a user with increased accuracy.

The calculating unit 13 and the transmitting unit 14 shown in FIG. 1 may be implemented inside the apparatus shown in FIG. 3. The calculating unit 13 implemented inside the apparatus may measure the PWV of a user based on the measurement results provided by the PCG sensor 111, the PPG sensor 112, the pressure sensor 113 and the ECG sensor (not shown) which are implemented outside the apparatus. In addition, the transmitting unit 14 implemented inside the apparatus may transmit the PWV calculated by the calculating unit 13 to an external destination.

Although not shown in FIG. 3, the apparatus according to the present embodiment may further include a display unit. In this case, the display unit may display a PWV calculated by the calculating unit 13 so that a user can check the condition of his or her blood vessels.

A user can easily hold the apparatus according to the present embodiment by the hand, place the apparatus on the skin near the heart, and measure his or her PWV. In addition, since the pressure sensor 113 is mounted on the apparatus, changes in the PPG caused by the grip strength of a user can be compensated for.

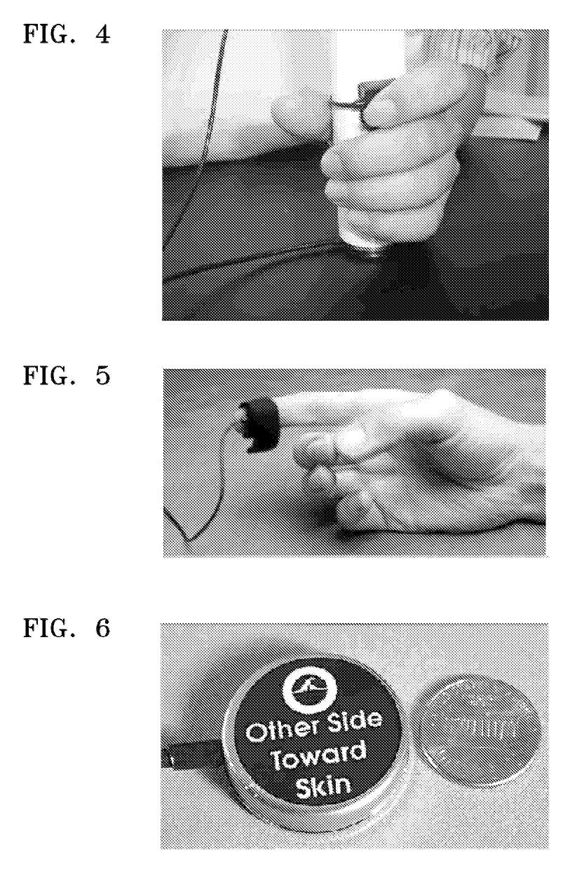

FIG. 4 shows an example of the apparatus of FIG. 3 in which a plurality of sensors are integrated. FIG. 5 shows an example of a PPG sensor included in the apparatus of FIG. 4. FIG. 6 shows an example of a PCG sensor included in the apparatus of FIG. 4.

Referring to FIGS. 4 through 6, the PPG sensor of FIG. 5 is mounted on a cylindrical surface of the apparatus of FIG. 4, and the PCG sensor of FIG. 6 is mounted on a bottom surface of the apparatus of FIG. 4. In this case, the PPG sensor of FIG. 5 may use a transducer (TSD) 200 manufactured by Biopac Corporation, and the PCG sensor of FIG. 6 may use a TSD 108 manufactured by Biopac Corporation.

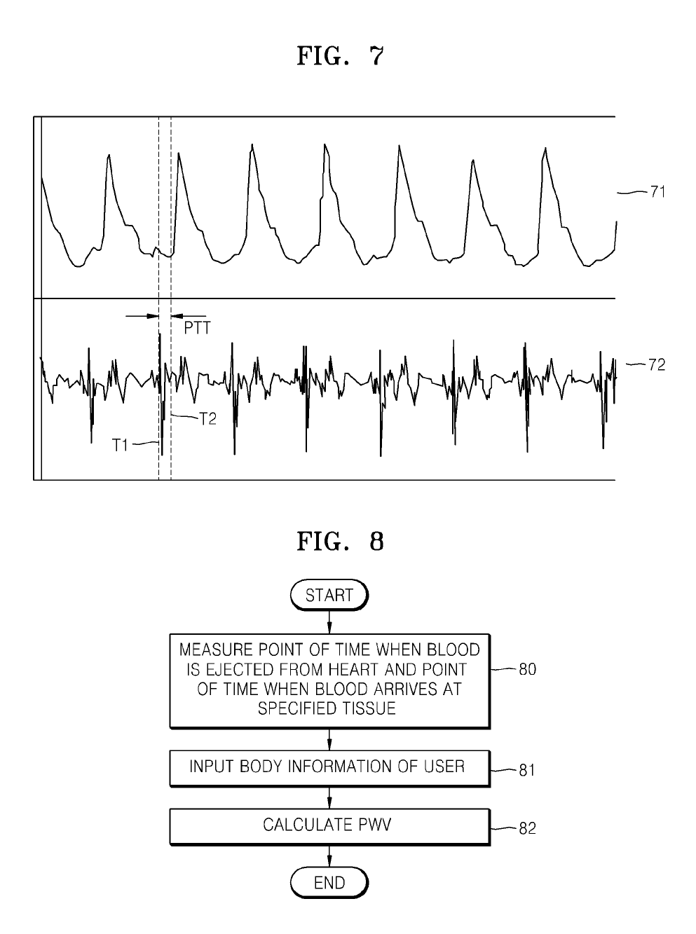

FIG. 7 shows waveforms of a PPG 71 and a PCG 72 measured by the apparatus of FIG. 4.

Referring to FIG. 7, a peak of the PCG 72 indicates a point of time when the heart begins to contract and when the first heart sound is generated. A first point of time T1 when the PCG 72 peaks is when blood is ejected from the heart. A second point of time T2 when the PPG 71 begins to peak is when the blood ejected from the heart arrives at a specified tissue. Therefore, the difference between the first point of time T1 and the second point of time T2 is a PTT.

A PWV can be calculated using the PTT calculated based on the waveforms of FIG. 7. The PWV calculated as described above is similar to a PWV measured using each of a conventional PPG sensor and a conventional PCG sensor.

FIG. 8 is a flowchart illustrating a method of measuring a PWV according to an embodiment of the present invention.

Referring to FIG. 8, the method according to the present embodiment includes operations sequentially performed by the apparatus shown in FIG. 1. Therefore, technical features of the apparatus of FIG. 1 described above apply to the method according to the present embodiment even though their description is omitted or simplified below.

In operation 80, the biological signal measuring sensor 11, into which a number of sensors are integrated, measures a point of time when blood is ejected from the heart of a user and a point of time when the blood arrives at a specified tissue. Specifically, the biological signal measuring sensor 11 may measure a heart sound generated when valves of the heart are opened or closed in order to measure a point of time when blood is ejected from the heart. In addition, the biological signal measuring sensor 11 may measure the amount of light absorbed by a specified tissue in order to measure a point of time when the blood arrives at the specified tissue. Alternatively, the biological signal measuring sensor 11 may measure the action potential of heart muscle cells, which are caused by a heartbeat, in order to measure a point of time when blood is ejected from the heart. In addition, the biological signal measuring sensor 11 may measure the amount of light absorbed by a specified tissue in order to measure a point of time when the blood arrives at the specified tissue.

In operation 81, a user inputs his or her body information.

In operation 82, the calculating unit 13 calculates a PWV according to the systole and diastole by using the measurement results and the input body information.

The method according to the present embodiment may further include an operation of measuring the grip strength of the user.

In addition, the method according to the present embodiment may further include an operation of transmitting a calculated PWV to an external destination.

In addition to the above described embodiments, embodiments of the present invention can also be implemented through computer readable code/instructions in/on a medium, e.g., a computer readable medium, to control at least one processing element to implement any above described embodiment. The medium can correspond to any medium/media permitting the storing and/or transmission of the computer readable code.

The computer readable code can be recorded/transferred on a medium in a variety of ways, with examples of the medium including recording media, such as magnetic storage media (e.g., ROM, floppy disks, hard disks, etc.) and optical recording media (e.g., CD-ROMs, or DVDs), and transmission media such as carrier waves, as well as through the Internet, for example. Thus, the medium may further be a signal, such as a resultant signal or bitstream, according to embodiments of the present invention. The media may also be a distributed network, so that the computer readable code is stored/transferred and executed in a distributed fashion. Still further, as only an example, the processing element could include a processor or a computer processor, and processing elements may be distributed and/or included in a single device.

As described above, an apparatus for measuring a biological signal according to an embodiment of the present invention may include a first surface having a first sensor, which is attached to a predetermined part of a user's body and measures a first biological signal generated by the predetermined part, and a second surface having a second sensor which is attached to the user's finger and measures a second biological signal generated by the finger. Therefore, the user can easily check his or her health condition without being limited by time or place.

An apparatus for measuring a PWV according to an embodiment of the present invention includes a biological signal measuring sensor in which a first sensor and a second sensor are integrated, an input unit, and a calculating unit. The first sensor of the biological signal measuring sensor is attached to the skin near the heart of a user and measures the systole and diastole caused by a heartbeat. In addition, the second sensor of the biological signal measuring sensor is attached to the user's finger and measures changes in the blood volume in blood vessels near the finger which are caused by a heartbeat. The input unit receives body information of the user, and the calculating unit calculates a PWV according to the systole and diastole by using the measurement results of the biological signal measuring sensor and the body information provided by the input unit. Therefore, a user can check the condition of his or her blood vessels without being limited by time or place.

Since a user can carry the apparatus and check the condition of his or her blood vessels, the user can recognize the influence of certain foods or exercises on the condition of his or her blood vessels. Thus, the user can determine which food or exercise is helpful for his or her health.

The biological signal measuring sensor of the apparatus according to the embodiment of the present invention may further include a pressure sensor in order to remove noise caused by the force which is applied by a user to grip the apparatus, that is, grip strength, and to ensure the user maintains a grip strength greater than a predetermined level.

The apparatus according to the embodiment of the present invention may further include a transmitting unit which transmits a measured PWV to an external destination. Based on the measured PWV, a medical center, for example, can remotely diagnose the condition of a patient's blood vessels and determine how much arteriosclerosis the patient has developed.

While aspects of the present invention has been particularly shown and described with reference to differing embodiments thereof, it should be understood that these exemplary embodiments should be considered in a descriptive sense only and not for purposes of limitation. Any narrowing or broadening of functionality or capability of an aspect in one embodiment should not considered as a respective broadening or narrowing of similar features in a different embodiment, i.e., descriptions of features or aspects within each embodiment should typically be considered as available for other similar features or aspects in the remaining embodiments.

Thus, although a few embodiments have been shown and described, it would be appreciated by those skilled in the art that changes may be made in these embodiments without departing from the principles and spirit of the invention, the scope of which is defined in the claims and their equivalents.

What is claimed is:

1. An apparatus for measuring a biological signal, the apparatus comprising:
a first surface having a first sensor which is attached to a predetermined part of a user's body and measures a first biological signal generated by the predetermined part; and
a second surface having a second sensor which is attached to the user's finger and measures a second biological signal generated by the finger.

2. The apparatus of claim 1, wherein the first surface is included in a first member, and the second surface is included in a second member, which is coupled to the first member, and comprises grooves formed in the second member so that the user can hold the second surface by the hand.

3. The apparatus of claim 1, wherein the first sensor is attached to the skin near the heart of the user and measures the systole and diastole caused by a heartbeat.

4. The apparatus of claim 1, wherein the second sensor is attached to the user's finger and measures changes in the blood volume in blood vessels near the finger which are caused by a heartbeat.

5. A biological signal measuring sensor comprising:
a first sensor to be attached to the skin near the heart of a user and measure the systole and diastole caused by a heartbeat; and
a second sensor to be attached to the user's finger and measure changes in the blood volume in blood vessels near the finger which are caused by a heartbeat,
wherein the first and second sensors are integrated in the biological signal measuring sensor.

6. The sensor of claim 5, wherein the biological signal measuring sensor is implemented as a pole of a predetermined shape, the first sensor is mounted on a bottom surface of the biological signal measuring sensor, and the second sensor is mounted on a cylindrical surface of the biological signal measuring sensor.

7. The sensor of claim 6, wherein the second sensor is mounted in grooves which are formed on the cylindrical surface of the biological signal measuring sensor.

8. The sensor of claim 6, further comprising a pressure sensor to measure the grip strength of the user, wherein the pressure sensor is mounted in the grooves which are formed on the cylindrical surface of the biological signal measuring sensor.

9. The sensor of claim 5, wherein the first sensor is a phonocardiogram (PCG) sensor measuring a heart sound which is generated when valves of the heart are opened or closed.

10. The sensor of claim 5, wherein the first sensor is an electrocardiogram (ECG) sensor measuring the action potential of heart muscle cells which is caused by a heartbeat.

11. The sensor of claim 5, wherein the second sensor is a photoplethysmography (PPG) sensor attached to the user's finger and measuring changes in absorbance in the blood vessels near the finger.

12. The sensor of claim 5, wherein the first sensor is mounted on a surface of the biological signal measuring sensor, and the second sensor is mounted on the other surface of the biological signal measuring sensor.

13. An apparatus for measuring a pulse wave velocity (PWV), the apparatus comprising:
a biological signal measuring sensor in which a first sensor and a second sensor are integrated, the first sensor being attached to the skin near the heart of the user and measuring the systole and diastole caused by a heartbeat, and the second sensor being attached to the user's finger and measuring changes in the blood volume in blood vessels near the finger which are caused by a heartbeat;
an input unit to receive a user's body information; and
a calculating unit to calculate a PWV according to the systole and diastole of the user by using measurement results of the biological signal measuring sensor and the body information input to the input unit.

14. The apparatus of claim 13, wherein the biological signal measuring sensor further comprises a pressure sensor to measure the grip strength of the user.

15. The apparatus of claim 13, further comprising a transmitting unit to transmit the PWV calculated by the calculating unit to an external destination.

16. The apparatus of claim 13, wherein the first sensor measures the systole and diastole in order to measure a point of time when blood is ejected from the heart, and the second sensor measures changes in the blood volume in blood vessels near the finger in order to measure a point of time when the blood arrives at the blood vessels near the finger.

17. The apparatus of claim 13, wherein the first sensor is a PCG sensor measuring a heart sound which is generated when valves of the heart are opened or closed or an ECG sensor measuring the action potential of heart muscle cells which is caused by the heartbeat, and the second sensor is a PPG sensor attached to the user's finger and measuring changes in absorbance in the blood vessels near the finger.

18. The apparatus of claim 13, wherein the biological signal measuring sensor is implemented as a pole of a predetermined shape, the first sensor is mounted on a bottom surface of the biological signal measuring sensor, and the second sensor is mounted on a cylindrical surface of the biological signal measuring sensor.

19. The apparatus of claim 18, wherein the second sensor is mounted in grooves which are formed on the cylindrical surface of the biological signal measuring sensor.

20. A method of measuring a PWV, the method comprising:
measuring a point of time when blood is ejected from the heart of a user and a point of time when the blood arrives at a specified tissue by using a biological signal measuring sensor in which a plurality of sensors are integrated;
inputting body information of the user; and
calculating a PWV according to the systole and diastole of the user by using measurement results and the input body information.

21. The method of claim 20, further comprising measuring the grip strength of the user.

22. A computer-readable medium having a computer readable code to implement a method of measuring a PWV, the method comprising:
measuring a point of time when blood is ejected from the heart of a user and a point of time when the blood arrives at a specified tissue by using a biological signal measuring sensor in which a plurality of sensors are integrated;
inputting body information of the user; and
calculating a PWV according to the systole and diastole of the user by using measurement results and the input body information.
